(19) United States
(12) Patent Application Publication
(10) Pub. No.: US 2021/0046085 A1
(43) Pub. Date: Feb. 18, 2021

(54) TREATMENT OF ADVANCED ESTROGEN RECEPTOR POSITIVE BREAST CANCER

(71) Applicant: Pantarhei Oncology B.V., Zeist (NL)

(72) Inventors: Herman Jan Tijmen COELINGH BENNINK, Zeist (NL); Carole VERHOEVEN, Zeist (NL)

(21) Appl. No.: 17/086,101

(22) Filed: Oct. 30, 2020

Related U.S. Application Data
(63) Continuation of application No. PCT/EP2019/061143, filed on May 1, 2019.

(30) Foreign Application Priority Data
May 2, 2018 (EP) 18170397.6

(51) Int. Cl. A61K 31/565; A61K 45/06; A61P 35/00

(52) U.S. Cl. CPC A61K 31/565; A61P 35/00; A61K 45/06

(57) ABSTRACT

The invention relates to the treatment of advanced estrogen receptor positive breast cancer in a subject who has been treated with an estrogen activity suppressor selected from a selective estrogen receptor modulator (SERM), an aromatase inhibitor and an anti-estrogen, said treatment comprising administration of an estriol component after the treatment with an estrogen activity suppressor has been discontinued, said estriol component being selected from estriol, prodrugs of estriol and combinations thereof.

### CROSS-REFERENCE TO RELATED APPLICATIONS

This application is a continuation of International Application No. PCT/EP2019/061143, filed May 1, 2019, which claims the benefit of and priority to European Application No. 18170397.6 filed May 2, 2018, both of which are hereby incorporated by reference herein in their entireties.

### FIELD OF THE INVENTION

The present invention relates to the field of breast cancer treatment. More particularly, the invention relates to the treatment of advanced estrogen receptor positive breast cancer in a subject who has been treated with an estrogen activity suppressor selected from a selective estrogen receptor modulator (SERM), an aromatase inhibitor and an anti-estrogen, said treatment comprising administration of an estriol component selected from estriol, prodrugs of estriol and combinations thereof, within 12 weeks after the treatment with an estrogen activity suppressor has been discontinued; wherein doses of the estriol component are administered uninterruptedly during a period of at least 2 weeks in dosages equivalent to a daily oral dosage of at least 10 mg estriol.

### BACKGROUND ART

Breast cancer is one of the leading causes of cancer mortality among Western women, and is predicted to become a leading cause of cancer death in Oriental women in countries such as Japan in the near future. The American Cancer Society estimates that 1 in 9 women face a lifetime risk of this disease, which will prove fatal for about one-quarter of those afflicted with the disease. Breast tumours are known to be estrogen-sensitive, meaning that the formation and growth of such tumours is stimulated by estrogens such as 17beta-estradiol. 17beta-estradiol is an estrogen that is endogenous to the human body and that is found in both females and males. Estrogens are known to increase the risk of breast tumours by inducing an estrogen receptor (ER) mediated increase in the frequency of breast cell division (proliferation). Cell division is essential in the complex process of genesis of human cancer since it per se increases the risk of genetic error, particularly genetic errors such as inactivation of tumour suppressor genes.

An important element of the treatment of estrogen-sensitive tumours is the suppression of undesirable estrogen-induced effects. Estrogen induced effects can be suppressed or even eliminated by administering an estrogen activity suppressor such as a selective estrogen receptor modulator (SERM), an aromatase inhibitor or an anti-estrogen.

A commonly used therapy to block estrogen receptor sites involves the administration of anti-estrogens. Anti-estrogens are a class of chemicals which prevent estrogens from eliciting their full response in target tissues. An example of such a compound is fulvestrant, which is a pure anti-estrogen since it degrades the estrogen receptor.

Selective estrogen receptor modulators (SERMs) are another class of estrogen activity suppressors that are commonly used in the treatment of estrogen-sensitive cancers. Tamoxifen (TAM) is an example of a SERM. Unlike anti-estrogens, SERMs exhibit both estrogen antagonist and agonist properties.

Aromatase Inhibitors (Als) work by blocking the production of estrogens. There are two types of aromatase inhibitors approved to treat breast cancer: irreversible steroidal inhibitors, such as exemestane, which forms a permanent and deactivating bond with the aromatase enzyme (the enzyme responsible for the synthesis of estrogens), and non-steroidal inhibitors, such as anastrozole and letrozole which inhibit the synthesis of estrogen via reversible competition for the aromatase enzyme.

Treatment of breast cancer by administering an estrogen activity suppressor (endocrine therapy) is often highly effective, but its usefulness is limited by common intrinsic and acquired resistance. Multiple mechanisms responsible for endocrine resistance have been proposed and include deregulation of various components of the ER pathway itself, alterations in cell cycle and cell survival signaling molecules, and the activation of escape pathways that can provide tumours with alternative proliferative and survival stimuli.

Thus, despite the benefits of using estrogen activity suppressor in the treatment of ER-positive breast cancer, resistance to treatment eventually occurs in a large number of patients (A. A. Larionov and W. R. Miller, Future Oncology, vol. 5, no. 9, pp. 1415-1428, 2009). Clinically, resistance can manifest itself as relapse or cancer recurrence during or after completion of drug therapy, following surgery or in rare cases after complete pathological response (elimination of all cancer tissue). Alternatively, in the neoadjuvant setting, resistance can be observed as clinical progression of primary disease, usually constituting an increase in primary tumour size or disease spread to regional nodes or beyond to more distant metastatic sites. Pathological changes such as increased tumour grade or increased proliferation are indicators of potential resistance to therapy. In the neoadjuvant setting, resistance occurs as either a primary lack of response early in treatment, implying innate resistance, or later following a period of response, suggesting acquired resistance. It has been suggested that as many as 40-50% of all ER-positive patients treated with estrogen activity suppressor will eventually relapse (C. X. Ma, C. G. Sanchez, and M. J. Ellis, Oncology, vol. 23, no. 2, pp. 133-142, 2009).

The current clinical practice, when confronted with a breast tumour having developed resistance to estrogen activity suppressor, is to initiate treatment with chemotherapy drugs. These powerful drugs, however, have very detrimental side effects. Therefore, there is a great need for alternative treatments for advanced estrogen receptor positive breast cancers that have less damaging side effects.

Estriol is one of the four natural human estrogens. It was discovered in the urine of pregnant women in 1930. In humans, estriol is one of the metabolic end-products of estradiol. Estradiol is reversibly oxidized to estrone and both estradiol and estrone can be (irreversibly) converted to estriol in the liver. Typical circulating levels of estriol are 7 pg/mL in the follicular phase and 11 pg/mL in the luteal phases, corresponding to production rates of 14 and 23 pg/day respectively. Levels found in postmenopausal women are 6 pg/mL. Estriol is the main estrogen of pregnancy. During pregnancy levels are approximately 1000 times higher (11-14 ng/mL) as compared to normal non-pregnant levels. Estriol has lower estrogenic activity than estradiol. It has a low affinity for binding to the Sex Hormone Binding Globulin, so most of the circulating estriol is available for biological activity.

Estriol has been marketed for several decades in Europe for the treatment of postmenopausal complaints under different brand names, including Synapause®, Ovestin®, Evalon® and Femastin®. It is available in tablets for oral treatment and as a vaginal cream.

E3 is reported to have a very short half-life after oral administration. Values between 1.5 hour (Summary of Product Characteristics of Synapause®) and 9-10 hours (Pharmacokinetics of estrogens and progestogens, Maturitas (1990), 12:171-197) have been reported. E3 is almost completely conjugated in the intestine to glucuronides (80-90%) and sulfates (10-20%), only 1-2% reaches the circulation. For that reason, the vaginal route is the preferred route of administration for clinical use.

Lemon H.M. has reported that estriol provides a protective effect against the development of breast cancer (Estriol and prevention of breast cancer, The Lancet (1973), 10:546-47). Estriol was found to be the most active protective estrogen yet tested against neoplasms induced by 20 mg oral 7,12-dimethyl-benzanthracene (DMBA) or by 50 mg procarbazine (PC) in Sprague-Dawley female rats. The author expressed the hope that these observations will provide a basis for extended clinical trials of estriol in premenopausal Caucasian women. Candidates would include those with a familial history of breast cancer, those with genetically impaired estrogen hydroxylation, those with precancerous breast changes, or those who chose to avoid pregnancy. Reference is made to an ongoing clinical trial in breast cancer, in which 5.0 mg estriol per day is well tolerated for as long as eleven months.

In 1982, Englund et al. assessed the bioavailability of estriol after oral administration of 6 mg or 12 mg in women. Estriol was rapidly absorbed when given orally, with plasma peaks after 15 to 60 minutes followed by a gradual decrease to low levels within 3 to 4 hours. Peak levels after oral administration of 6 mg estriol ranged between 80-220 pg/mL whereas peak levels after administration of 12 mg ranged between 150-490 pg/mL.

In 1984, Heimer and Englund evaluated the absorption of a single oral dose of 12 mg estriol in postmenopausal women and they especially assessed whether enterohepatic circulation might play a role in the pharmacokinetics of estriol. After oral administration, they found an initial increase in estriol levels (500-1000 pmol/L, i.e. 144-288 pg/mL) which lasted for about four hours followed by a second and possible third increase immediately after the meals.

Lippman et al. (Effects of Estrone, Estradiol, and Estriol on Hormone responsive Human Breast Cancer in Long-Term Tissue, Cancer Research (1977), 37, 1901-1907) compared the effects of estrone, estradiol, and estriol on MCF-7 human breast cancer. In this estrogen-responsive cell line, all three estrogens were capable of inducing equivalent stimulation of amino acid and nucleoside incorporation. Estriol was capable of partially overcoming anti-estrogen inhibition with Tamoxifen (ICI 46474), even when anti-estrogen is present in 1000-fold excess. Anti-estrogen effects were completely overcome by 100-fold less estriol. All three steroids were found to bind to a high-affinity estrogen receptor found in these cells. The apparent dissociation constant was lower for estradiol than for estrone and estriol, but all three bind to an equal number of sites when saturating concentrations are used. The authors conclude that estriol can bind to estrogen receptor and stimulate human breast cancer in tissue culture and that their data do not support an anti-estrogenic role for estriol in human breast cancer.

The effect of estriol on growth of MCF-7 human breast cancer cell lines has also been investigated by Diller et al. (Effects of estriol on growth, gene expression and ERE activation in human breast cancer cell lines. Maturitas (2014) 77, 336-343). It was found that estriol acted as a potent estrogen and exerted a mitogenic effect on T-47D and MCF-7 cells at concentrations of 10⁻⁹M (288 pg/ml) and higher. With regard to activation of an estrogen response element (ERE) in breast cancer cells, effects of estriol were visible at 10⁻¹⁰ M. The same concentrations of estriol activated expression of the estrogen-responsive gene PR and of the proliferation genes cyclin A2, cyclin B1, Ki-67, c-myc and b-myb, providing molecular mechanisms underlying the observed growth increase. The authors conclude: Like E2 (estradiol), low levels of E3 were able to trigger a robust estrogenic response in breast cancer cells. Thus, our data suggest caution regarding use of E3 by breast cancer survivors.

The use of estriol as a potential agent in the treatment of cancer has been investigated by Girgert et al. (Inhibition of GPR30 by estriol prevents growth stimulation of triple-negative breast cancer cells by 17β-estradiol, BMC Cancer (2014) 14:935) showed that GPR30 is involved in growth stimulation of triple-negative breast cancer by 17β-estradiol. Estriol effectively inhibited signal transduction of GPR30 and successfully prevented growth promotion by 17β-estradiol. These results clearly show that a pharmacological inhibition of GPR30 is a promising targeted treatment option for triple-negative breast cancer. However, the authors conclude that the concentrations of estriol needed for sufficient growth inhibition are unfortunately unphysiologically high and that consequently there is a need for developing more effective inhibitors for GPR30.

Horn et al. (Randomized study comparing chemotherapy with and without estrogen priming in advanced breast cancer”, Int. J. of Oncology (1994); 4(2), 499-501) report a study in which women with estrogen or progesterone receptor positive advanced breast cancer received chemotherapy with and without estrogen priming. Patients refractory to prior endocrine therapy who entered the study had received primarily tamoxifen. Estrogen priming consisted of oral tablets containing 2 mg estradiol and 1 mg estriol (Estrofem) given twice daily beginning on day 1 and continuous with chemotherapy from day 7 on.

WO 2007/038636 mentions the combined use of estriol and secondary active agents (e.g. progesterone) in the treatment of patients exhibiting symptoms of a neurodegenerative disease.

### SUMMARY OF THE INVENTION

The present invention provides a useful alternative therapy for treating advanced estrogen receptor positive breast cancers that have become resistant to an estrogen activity suppressor or for treating advanced estrogen positive breast cancers in subjects who have rejected treatment with estrogen activity suppressor due to unacceptable side effects.

More particularly, the invention provides a treatment of advanced estrogen receptor positive breast cancer in a subject who has been treated with an estrogen activity suppressor selected from a selective estrogen receptor modulator (SERM), an aromatase inhibitor and an anti-estrogen, said treatment comprising administration of an estriol component to said subject within 12 weeks after the treatment with an estrogen activity suppressor has been discontinued, said estriol component being selected from estriol, prodrugs of estriol in the form of estriol derivatives wherein the hydrogen atom of at least one of the hydroxyl groups has been substituted by an acyl radical of a hydrocarbon carboxylic, sulfuric acid, sulfonic acid or sulfamic acid of 1-25 carbon atoms; or combinations thereof, and combinations thereof; wherein doses of the estriol component are administered uninterruptedly during a period of at least 2 weeks in dosages equivalent to a daily oral dosage of at least 10 mg estriol.

The inventors have found that administration of an estriol component to a subject suffering from breast cancer, and who has been treated with an estrogen activity suppressor, can have an unexpected favourable impact on tumour progression, and in some cases may even induce tumour regression. In addition, due to its estrogenicity, administration of the estriol components to these subjects has an advantageous effect on Quality Of Life (QOL). Thus, treatment with the estriol component can suitably be used to delay treatment with chemotherapy drugs and to eliminate hypoestrogenicity that was caused by the preceding treatment with estrogen activity suppressor.

This surprising finding stems from the observation by the inventors that an estriol component can be administered at high doses (e.g. equivalent to more than 10 mg estriol p.o. per day) to breast cancer patients without generating the unacceptable side effects usually observed for high doses of other estrogens. Furthermore, the treatment of the invention has a positive impact on the hypo-estrogenic side effects induced by previous treatment with estrogen activity suppressors. Examples of undesirable side-effect that can be remedied by the present treatment include mood disturbances (depression/irritability), hot flushes, arthralgia, vulvovaginal complaints, sleep disturbances, cognition problems, memory loss and bone loss.

The present treatment may employ oral, mucosal (such as sublingual, sublabial, buccal, intranasal), transdermal, parenteral (such as i.v.) or subcutaneous administration of the estriol component.

### DETAILED DESCRIPTION OF THE INVENTION

### Definitions

The term “estriol component”, as used herein, encompasses estriol as well as prodrugs of estriol in the form of estriol esters that are estriol derivatives wherein the hydrogen atom of at least one of the hydroxyl groups has been substituted by an acyl radical of a hydrocarbon carboxylic, sulfuric acid, sulfonic acid or sulfamic acid of 1-25 carbon atoms.

The term “estriol” refers to estra-1,3,5(10)-triene-3,16α,17β-triol. The term estriol also encompasses hydrates of this estrogen.

Whenever a “dose” or a “daily dose” is defined in terms of “estriol equivalent”, what is meant is that the dosage administered is equivalent to an orally administered estriol monohydrate dose as specified.

As used herein, the terms “advanced breast cancer” refers to locally advanced breast cancer (breast cancer that has progressed locally but there are no signs that the cancer has spread beyond the breast region) and/or metastatic breast cancer (breast cancer that has spread from its site of the origin to other parts of the body).

The qualification that a breast cancer has “acquired resistance to an estrogen activity suppressor” means that the treatment of the breast cancer with said estrogen activity suppressor is no longer effective as evidenced by a “progressive disease” categorization in accordance with the revised RECIST guideline (New response evaluation criteria in solid tumours: Revised RECIST guideline (version 1.1), Eur. J. Cancer (2009), 45, 228-247).

According to this guideline, the assessment is performed by comparing images of the tumour(s) at different stages during treatment. As further detailed in Section 4.3.1 of the revised RECIST guideline, the target lesions are classified into one of the following categories;
complete response (CR);
partial response (PR);
progressive disease (PD);
stable disease (SD).

In accordance with the present invention, administration of the estriol component commences within 12 weeks after the treatment with an estrogen activity suppressor has been discontinued. The time period between discontinuation of the treatment with an estrogen activity suppressor and the beginning of the administration of the estriol component equals the number of days that has lapsed between the first day on which the estrogen activity suppressor has not been administered according to the relevant protocol and the day on which the estriol component is first administered.

Methods of Treatment

The inventors have unexpectedly found that after oral administration of estriol (5-40 mg), plasma levels of estriol remained fairly constant over 24 hours, due to enterohepatic circulation. After oral administration of 20 mg or 40 mg estriol, plasma levels of estriol were still significant after 72 hours, indicating that orally administered estriol has a much longer half-life than is currently assumed. This finding means that once daily oral administration of estriol is an attractive option.

In accordance with a preferred embodiment, the present treatment comprises uninterrupted administration of doses of the estriol component during a period of at least 2 weeks, preferably during a period or at least 4 weeks, in dosages equivalent to a daily oral dosage of 15 to 150 mg estriol, even more preferably equivalent to a daily oral dosage of 20 to 120 mg estriol and most preferably equivalent to a daily oral dosage of 40 to 100 mg.

The amounts needed to be effective differ from individual to individual and are determined by factors such cancer type and stage, body weight, route of administration and the efficacy of the particular estrogenic substance used.

Administration of the estriol component in accordance with the present invention preferably commences within 8 weeks, more preferably within 4 weeks after the treatment with an estrogen activity suppressor has been discontinued.

In one embodiment of the invention, the treatment comprises administration of the estriol component to an oophorectomized or post-menopausal female subject who has decided to discontinue the treatment with an estrogen activity suppressor. Unacceptable symptoms of hypoestrogenicity are the main reason for subjects to discontinue treatment with estrogen activity suppressor. Administration of the estriol component in accordance with the present invention quickly removes these unacceptable symptoms and generally has a favourable impact on the progression of the breast cancer.

In another, particularly preferred embodiment of the present invention, the treatment comprises administration of the estriol component to a subject whose breast cancer has acquired resistance to said estrogen activity suppressor.

It is important to note that, prior to the present invention, the generally accepted practice for treating advanced estrogen receptor positive breast cancer that has become resistant to an estrogen activity suppressor was to avoid administering any kind of estrogen.

For example in a 2013 information booklet about menopause (accessible at: https://www.fda.gov:80/FDAgov/ForConsumers/ByAudience/ForWomen/ucm118627.htm), the FDA strongly warns against taking estrogen-containing medicines “if you have or have had certain cancers such as breast cancer or uterine cancer”.

Similarly, the Patient Information Leaflet for the estrogen-only product Premarin states “Do not take Premarin [ . . . ] if you have or have ever had breast cancer, or if you are suspected of having it”.

In a 2009 publication, Ellis et al. (Matthew J. Ellis et al; 2009, JAMA, 302(7): 774-780) report that estrogen treatment has an adverse effect on Quality of Life: as can be read in the Results section, under the paragraph “Quality of life analysis”,
“a significant increase in severity of side effects from baseline to follow-up was observed overall (0.47 to 0.80; P<0.001), but the change was not significantly different by treatment arm (0.47-0.70 in 6-mg arm vs. 0.46-0.92 in 30-mg arm; P=0.10)”.

Further, in the Discussion section of the publication, Ellis et al. state:
“We also observed that intense estradiol side effects have an adverse effect on QOL which are mitigated by lowering the estradiol dose.”

Thus it goes against this widely accepted doctrine to treat subjects with advanced estrogen receptor positive breast cancer with estrogenic estriol component.

Without wishing to be bound by theory, the unexpected benefits (and absence of undesired estrogenic effects) are believed to be associated with the special properties of the estriol component.

So far, estrogen administration in the context of breast cancer treatment has been constrained in terms of acceptable doses because of a number of side effects (such as, for example, nausea, or even thromboembolic and cardiovascular events in case of oral administration). The present applicant has demonstrated (among others, in Example 2), that an estriol component can be administered in high doses without generating the side effects usually observed with the administration of high doses of other estrogens.

In addition, the applicant has demonstrated (among others, in Example 1), that administration of an estriol component is surprisingly capable of delaying tumor growth.

Further, the present treatment is very effective in counteracting the hypo-estrogenism that is observed in breast cancer patients who have been treated with estrogen activity suppressors. The symptoms of hypo-estrogenism can be very severe and include mood disturbances (depression/irritability), hot flushes, arthralgia, vulvovaginal complaints, sleep disturbances, cognition problems, memory loss and bone loss.

A very important benefit of the second line treatment according to the present invention in comparison with ordinary second line treatment with chemotherapy drugs lies in the fact that contrary to the latter treatment, the present treatment does not have an adverse effect on Quality of Life (QOL) of the subject. As a matter of fact, as explained above, due to the fact that the present treatment counteracts hypo-estrogenism that is induced during the first line treatment, QOL of subjects is actually improved with the present treatment.

The quality of life of the subject is for example assessed with the Functional Assessment of Cancer Therapy-Breast (FACT-B) and Endocrine Subscale (FACT-ES) (version 4, see http://www.facit.org/FACITOrg/Questionnaires). More information on these assessments can be found in publications by Fallowfield et al. (British J. of Cancer, 2012, 106, p. 1062-1067) and by Webster et al. (Health and Quality of Life Outcomes, 2003, 1, p. 79).

Higher scores for the scales and subscales indicate better quality of life. A difference of 5 in the trial outcome index (TOI), a summation of the physical, functional and breast cancer concerns subscales, is considered to be the clinically relevant minimally important difference.

In a preferred embodiment, the quality of life of the subject treated by the therapy of the invention is accordingly improved during the course of the treatment by an increase of at least 5 in the TOI, preferably by an increase of at least 7 in the TOI, even more preferably an increase of at least 10 in the TOI.

In a particular embodiment of the invention, the estriol component is administered during a treatment period of at least 8 weeks, preferably at least 24 weeks, more preferably at least 1 year.

In a further embodiment, the tumour burden is monitored during the treatment period at regular intervals. If tumour burden decreases (CR or PR under the RECIST criteria) or remains stable (SD under the RECIST criteria), the treatment with estriol component is continued. If the tumour burden progresses (PD under the RECIST criteria) treatment with a chemotherapy drug is initiated.

According to a particularly preferred embodiment of the present treatment, administration of a chemotherapy agent is commenced when the monitoring shows that the tumour burden has increased.

In an even more preferred embodiment, administration of the estriol component is continued during administration of the chemotherapy agent, in particular if hypoestrogenic symptoms were improved during the earlier estriol treatment.

In a particular embodiment the estriol component is administered once daily in a convenient once-daily unit dose.

According to a particularly preferred embodiment, the present treatment comprises oral, sublingual, sublabial or buccal administration of the estriol component. Most preferably, the treatment comprise oral administration of the estriol component.

In the present treatment, the estriol component is preferably administered in an amount effective to achieve an estriol equivalent blood plasma trough concentration of at least 50 pg/mL, preferably of at least 100 pg/mL, more preferably at least 200 pg/mL, still more preferably at least 400 pg/mL and most preferably at least 800 pg/mL. As used herein, “trough levels” means the lowest concentration that a drug reaches before the next dose is administered.

Generally the resulting estriol equivalent blood plasma trough levels do not exceed 2000 pg/mL, preferably it does not exceed 1800 pg/mL, more preferably it does not exceed 1600 pg/mL, still more preferably it does not exceed 1400 pg/mL.

The safety of the estriol component is a key aspect of the present invention which makes it possible to administer this estrogenic component at much higher levels than other estrogens and thus renders the present treatment possible.

Yet another important aspect of the treatment of the invention is that, since estriol itself does not bind to SHBG, changes in plasma levels of SHBG do not influence the availability of estriol. This is by contrast to estradiol which binds to SHBG with high affinity of about 40% (Hammond GL at al., Climacteric. 2008; 11 Suppl 1:41-6). A direct consequence of this is that more of the administered estriol is available by comparison with an estradiol-based treatment where a significant part of the administered drug is bound to SHBG.

For reasons of convenience and also to achieve high compliance rates, the present treatment preferably utilises administration intervals of 1 day, 1 week or 1 month. Regimens that employ once daily oral, sublingual, buccal or sublabial administration of the estriol component are particularly preferred. Regimens that employ once daily oral administration of the estriol component are most preferred.

Patient Population

The subject treated in accordance with the invention is preferably a human, especially a female. Said subject suffers from locally advanced and/or metastatic (herein, “advanced”) breast cancer. Said breast cancer has an estrogen-receptor-positive status (ER+).

The present treatment is particularly effective if the subject is a post-menopausal female or a female who has undergone oophorectomy.

The subject undergoing treatment in accordance with the present invention preferably has previously been treated with tamoxifen and/or an aromatase inhibitor. In this embodiment, treatment with tamoxifen encompasses, for example, treatment with the drug Nolvadex™ and treatment with an aromatase inhibitor encompasses treatment with one or more of, for example, anastrosole (Arimidex™) letrozole (Femara™), exemestane (Aromasin™) and/or aminoglutethimide (Orimeten™).

In a particular embodiment, the subject has not received treatment with fulvestrant (7-alpha[9-(4,4,5,5,5-pentafluoropentylsulphinyl)nonyl]-estra-1,3,5(10)-triene-3, 17beta diol, marketed under the brand name Faslodex™), within 6 months of start of the treatment according to the invention.

Another aspect of the invention relates to an oral dosage unit comprising an oral chemotherapy drug, together with at least 10 mg, preferably 15-150 mg, more preferably 20-120 mg and most preferably 40-100 mg of the estriol component. Examples of oral chemotherapy drugs that may be employed in the oral dosage unit include capecitabine, cyclophosphamide, vinorelbine, methotrexate and combinations thereof.

Yet another aspect of the invention relates to a kit-of-parts comprising at least one dosage unit containing a chemotherapy drug and a plurality of oral dosage units containing at least 10 mg, preferably 15-150 mg, more preferably 20-120 mg and most preferably 40-100 mg estriol component. The chemotherapy drug is preferably provided in a dosage form that is suitable for intravenous administration. Examples of chemotherapy drugs that can be employed in the kit-of-parts include gemcitabine, docetaxel, paclitaxel, albumin-bound paclitaxel, cisplatin, carboplatin, doxorubicin, liposomal doxorubicin, epirubicin, eribulin, ixabepilone, cyclophosphamide, vinorelbine and combinations thereof.

According to a particularly preferred embodiment, the kit-of-parts contains instructions to co-administer the at least one dosage unit containing the chemotherapy drug and the dosage units containing the estriol component.

Examples of oral dosage units that may be employed in accordance with the present invention include tablets and capsules. Most preferably, the oral dosage unit is a tablet.

### EXAMPLES

### Example 1: In Vitro Study in Estrogen Deprived Cells

In order to assess the potential of an estriol component to inhibit the growth of estrogen deprived breast cancer cells, in vitro studies in long term estrogen deprived MCF7 breast cancer cells (LTED cells) were conducted. LTED cells were deprived of estrogen, making them a suitable in vitro model to study drug effects in women who have been using estrogen activity suppressors for a long time.

LTED cells were plated in 6-well plates at a density of 30,000 cells per well. The cells were maintained in phenol red free IMEM with 5% charcoal stripped FBS (DCC-FBS). On Day 3, the medium was replaced with fresh phenol red free IMEM with 5% DCC-FBS. The cells were then exposed to 6 different concentrations of estriol ranging from 10⁻¹² M to 10⁻⁵ M or to ethanol as vehicle control. The final vehicle concentration was 0.1% ethanol for all experiments. Each treatment was done in duplicate.

On Day 5, the medium was changed and on Day 7 plates were subjected to a cell count analysis.

The results are shown in Table 1.

TABLE 1

| | Cell number per well (×10⁶) |
|---|---|
| Control | 1.28 |
| 10⁻¹² M estriol | 1.17 |
| 10⁻¹¹ M estriol | 0.92 |
| 10⁻¹⁰ M estriol | 0.56 |
| 10⁻⁹ M estriol | 0.26 |
| 10⁻⁸ M estriol | 0.22 |
| 10⁻⁷ M estriol | 0.21 |
| 10⁻⁶ M estriol | 0.18 |
| 10⁻⁵ M estriol | 0.11 |

These results show that estriol is able to inhibit the growth of LTED cells. Peak inhibition is observed at 10⁻⁹M and no significant further inhibition is detected at higher estriol concentrations.

### Example 2: Estriol Plasma Levels

A single dose pharmacokinetics study was conducted to evaluate the pharmacokinetics of different oral dosages of estriol in postmenopausal women. In total 16 women were assigned to two different groups (Cohort A and Cohort B). Each Cohort received two estriol treatments. Women in Cohort A received single oral dosages of 5 and 20 mg E3 and women in Cohort B received single oral dosages of 10 and 40 mg estriol. A wash-out period of 7 days was taken into account between the treatments. Estriol plasma concentration were measured at regular interval up to 72 hours after administration.

The results of the test are summarised in Table 2.

TABLE 2

| | |
|---|---|
| Oral dose | Average plasma level (pg/mL) |

| (mg) | 0-24 hours | 24 hours | 48 hours | 72 hours |
|---|---|---|---|---|
| 5 | 28 | 17 ± 11 | 11 ± 15 | 2 ± 6 |
| 10 | 39 | 47 ± 67 | 12 ± 15 | 1 ± 2 |
| 20 | 103 | 64 ± 41 | 30 ± 10 | 8 ± 12 |
| 40 | 110 | 121 ± 113 | 52 ± 14 | 13 ± 8 |

The results of the study show that estriol is rapidly absorbed. Peak levels were observed at approximately 1-2 hours after dosing, followed by a gradual decrease 2-4 hours after dosing and a second increase that occurred 4-6 hours after dosing. The latter increase is attributed to enterohepatic circulation. Plasma levels of estriol remained fairly constant over the first 24 hours period and were still measurable up to 72 hrs post dosing.

In addition, this study shows that at high oral dose levels estriol is well tolerated. Comparable doses of other estrogens cause significant side effects such as nausea, impacting negatively on the QOL.

1. A method of treating advanced estrogen receptor positive breast cancer in a subject treated with an estrogen activity suppressor selected from a selective estrogen receptor modulator (SERM), an aromatase inhibitor and an anti-estrogen, the method comprising administering to the subject within 12 weeks of discontinuing treatment with the estrogen activity suppressor daily doses of an estriol component selected from estriol; prodrugs of estriol, wherein the hydrogen atom of at least one of the hydroxyl groups has been substituted by an acyl radical of a hydrocarbon carboxylic, sulfuric acid, sulfonic acid or sulfamic acid of 1-25 carbon atoms; and combinations thereof, wherein the doses are administered uninterruptedly for at least 2 weeks in an amount equivalent to a daily oral amount at least 10 mg estriol.

2. The method according to claim 1, wherein the doses are administered for at least 8 weeks.

3. The method according to claim 2, wherein the doses are administered for at least 24 weeks.

4. The method according to claim 3, the doses are administered for at least 1 year.

5. The method according to claim 1, wherein the subject has breast cancer having resistance to estrogen activity suppressor.

6. The method according to claim 1, wherein the subject is a post-menopausal human female or an oophorectomized human female.

7. The method according to claim 1, wherein the subject is a oophorectomized or post-menopausal female whose treatment with an estrogen activity suppressor was discontinued following the occurrence of hypo-estrogenic side effects.

8. The method according to claim 1, further comprising monitoring tumour burden during the treatment, and administration of a chemotherapy agent is commenced when the monitoring shows that the tumour burden has increased.

9. The method according to claim 8, wherein administration of the estriol component is continued during administration of the chemotherapy agent.

10. The method according to claim 1, wherein the doses are administered uninterruptedly for at least 2 weeks in an amount equivalent to 15 to 150 mg estriol.

11. The method according to claim 1, comprising oral, sublingual, buccal or sublabial administration of the estriol component.

12. The method according to claim 11, comprising oral administration of the estriol component.

13. The method according to claim 1, wherein the subject has not been treated with fulvestrant in the 6-months period preceding administration of the estriol component.

14. The method according to claim 1, wherein the estriol component is estriol.

15. An oral dosage unit, comprising (i) an oral chemotherapy drug selected from capecitabine, cyclophosphamide, vinorelbine, methotrexate and combinations thereof, and (ii) at least 10 mg of an estriol component selected from estriol, prodrugs of estriol in which the hydrogen atom of at least one of the hydroxyl groups has been substituted by an acyl radical of a hydrocarbon carboxylic, sulfuric acid, sulfonic acid or sulfamic acid of 1-25 carbon atoms; and combinations thereof.

16. A kit-of-parts comprising: (i) at least one dosage unit comprising a chemotherapy drug selected from gemcitabine, docetaxel, paclitaxel, albumin-bound paclitaxel, cisplatin, carboplatin, doxorubicin, liposomal doxorubicin, epirubicin, eribulin, ixabepilone, cyclophosphamide, vinorelbine and combinations thereof and (ii) a plurality of oral dosage units containing at least 10 mg estriol component selected from estriol, prodrugs of estriol in which the hydrogen atom of at least one of the hydroxyl groups has been substituted by an acyl radical of a hydrocarbon carboxylic, sulfuric acid, sulfonic acid or sulfamic acid of 1-25 carbon atoms; and combinations thereof.
